(19) United States
(12) Patent Application Publication
(10) Pub. No.: US 2026/0364014 A1
(43) Pub. Date: Nov. 19, 2026

(54) APPARATUS FOR TOPICAL DELIVERY OF CAPSULE POWDER

(71) Applicant: THE MATERIA COMPANY LIMITED, SAN GWANN (MT)

(72) Inventors: CHRISTIAN JOHANNES KILIAN DUCHOW, SAN GWANN (MT); MATTHEW VIDALIS, SAN GWANN (MT); RUSSELL FARRUGIA, SAN GWANN (MT)

(73) Assignee: THE MATERIA COMPANY LIMITED, SAN GWANN (MT)

(21) Appl. No.: 19/154,024

(22) PCT Filed: Feb. 7, 2024
(86) PCT No.: PCT/EP2024/053108

§ 371 Date: Aug. 6, 2025

(30) Foreign Application Priority Data
Feb. 7, 2023 (EP) 23155249.8

(51) Int. Cl. A61K 9/16

(52) U.S. Cl. CPC A61K 9/1635; A61K 9/1611; A61K 9/1617; A61K 9/1623

(57) ABSTRACT

An apparatus (1) for topical delivery of a powder composition to a body orifice or a mucous membrane thereof, from hard capsules (21) comprising a two-part housing, the container holder (3) and the capsule holder (2), optionally a detachable applicator (5), and a container (4) filled with propellant(s) suitable for immediately releasing only the gas form from the valve mechanism (25) upon actuation, at a pressure equal to the propellant's vapor pressure.

### FIELD OF THE INVENTION

The present invention relates to an apparatus for topical delivery of a powder composition to a body orifice or a mucous membrane thereof, from hard capsules (21). More specifically, it refers to a two-part housing comprising the container holder (3) and the capsule holder (2), a detachable applicator (5), optionally, and a container filled with propellant allowing only the gas form of the propellant to exit the valve mechanism upon actuation, as defined by the claims.

### BACKGROUND OF THE INVENTION

Topical administration is an attractive mode of therapy because it is painless, cost-effective and may reduce drug side-effects resulting from the first pass effect, i.e., hepatoxicity etc. The chemical agent can be directly applied to the affected area on the skin or membrane. Powder formulation has been used for topical applications. Nevertheless, their use is limited being difficult to apply, especially at mammals' orifices such as the oral, the rectal and the vaginal canal.

Aerosols have been used as an alternative to applicability into the mammal's cavities. The technology is based on a suspension or solution of fine powder particles solubilized in the liquified propellant. Heterogenous aerosol systems, where a homogenous suspension of powder exists inside a liquefied propellant vapor under pressure, are well known.

There is a need for efficient delivery of high delivered dose quantities, suitable for delivery into the mammal's orifices without the ‘cold freon effect’ caused by the high velocity blast and subsequent evaporation of the liquid propellant which can severely discomfort the patient and damage the skin and/or mucosal tissue, as propellant plume temperatures can drop down to −35 °C.

It is an object of the invention to deliver powder composition from hard capsule to a body orifice or body space without causing the “cold freon effect” being easy to use at home without use of laboratory equipment.

### SUMMARY OF THE INVENTION

The present invention relates to a powder delivery apparatus (1) for topical administration to a body orifice or a mucous membrane thereof, of powder from a hard capsule comprising a high pressure container (4) containing a propellant(s) in liquid and in gas form equipped with a valve mechanism (25) comprising a valve housing (22) and a valve stem (6), configured to be safely secured to the container holder (3), for immediately releasing only the gas form from the valve mechanism (25) upon actuation, at a pressure equal to the propellant's vapor pressure. The invention is defined in the appended claims.

The apparatus (1) comprises a) the container holder (3) comprising two compartments configured to communicate through an inlet gas orifice (7); wherein a substantially U-shaped compartment (9) is configured to be mounted on the capsule holder (2) by interlocking snapping means (13) on the external surface of the compartment and a breakage fin (8) mounted internally into the U-shaped compartment (9), and a second compartment (19) configured to retain the high pressure container valve mechanism (25) tip at the one end of the inlet gas orifice (7); b) a capsule holder (2) comprising an outlet nozzle (11), a compartment (20) configured to form a single mixing chamber (23) with the substantially U-shaped compartment (9) of the container holder (3) when interlocked for powder and propellant gas to be mixed; and an inlet opening (10) configured to retain at least partially a hard capsule (21). Preferably the container holder (3) and the capsule holder (2) are injected molded.

Within the scope of this invention the term “hard capsule” relates to a hard capsule made of two cylindrical shells, each closed off at one end. The upper part, called the capsule cap (16), has a slightly larger diameter than the lower part, called the capsule body (17). These two parts fit together perfectly to form a hard capsule (21). The dimensions and sizes of a hard capsule are standardized and well-known.

Within the scope of this invention is the use of the apparatus (1) for topical administration of the powder composition from a hard capsule.

Within the scope of this invention the term “topical administration” means application to a body surface such as the skin or mucous membranes. Preferably, the topical administration is topical administration to a body orifice or a mucous membrane thereof. More preferably, the topical administration is topical administration to the vagina. More preferably, the topical administration will effectively reach the vaginal cervix. In other aspects, the invention provides a topical administration of a powder composition to a body orifice, selected from the vaginal canal, the anus canal, the nostrils, the ear canal, the mouth, the tonsils, and to the mucous membranes thereof. Preferably, the invention is suitable for topical administration of a powder composition to a body orifice or a body hole. Mucous membranes lines cavities throughout a mammal's body that are exposed to particles from the outside.

The term “powder composition” typically contains an excipient, such as a binder, a disintegrant, a filler, a glidant, a preservative and may contain an active ingredient. The powder composition in hard capsules refers to a powder composition that is—amongst other features—preferably characterized as dry by its solvent content, which is preferably low enough to prevent the formation of aggregates that would reduce or inhibit the flowability of the powder. The residual of solvents and moisture content should be below 4%.

The apparatus (1) can optionally, comprise an applicator (5) configured to be mounted on the outlet nozzle (11). The applicator (5) can be a single-use or a multiple-use. In a preferred embodiment the applicator (5) is a disposable applicator (5). In a preferred embodiment the applicator (5) is characterized by thickness and length designed to match the dimensions of the target body orifice. The applicator (5) is equipped with a stopper (12).

Within the scope of the invention the high-pressure container (4) is containing a propellant(s). In a preferred embodiment the high-pressure container is containing a liquified propellant in liquid and in gas form. In a more preferred embodiment, the high-pressure container consisting essentially of a liquid propellant in liquid and in gas form.

The liquified propellant(s) expand substantially from a liquid form to a gas form when released to the atmosphere. The liquified propellant gas form is heavier than the atmospheric air. When a liquefied propellant is under pressure inside a container, it exists mainly as a liquid (liquid phase) and a fraction of the propellant will also be present in the head space of the container as a gas form (vapor phase). The gas form co-exists with the liquid form in the closed container and the two forms are in equilibrium. The vapor pressure will be equal to the equilibrium vapor pressure of the liquid propellant.

Within the scope of this invention, a propellant may be a mixture of propellants. In another embodiment the propellant(s) can be a mixture of propellants. In a further embodiment a propellant is a liquefied propellant.

In a preferred embodiment, the liquified propellant(s) include but are not limited to ethers, various hydrocarbons and hydrofluorocarbons. Liquefied propellants are high purity, non-corrosive, and the pressure obtained in a high-pressure container (4) will remain essentially constant over the duration of use of the product.

In a more preferred embodiment, the liquified propellant is selected from 1,1,1,2-Tetrafluoroethane, 1,3,3,3-Tetrafluoropropene and mixtures thereof 1,1,1,2-Tetrafluoroethane is commonly known as HFA 134a and 1,3,3,3-Tetrafluoropropene is commonly known as HFO1234ze. The preferred liquified propellant HFA and HFO are well-known for their non-flammability, low toxicity, poor solvency and low solubility. 1,1,1,2-Tetrafluoroethane has a boiling point of −26.3 °C. Furthermore, HFO-1234ze itself has zero ozone-depletion potential and has a boiling point of −19 °C.

Any suitable pressurized container can be mounted in the apparatus (1) of this invention. In a preferred embodiment the high pressurized container (4) is selected from a canister or a high-pressure glass container. In a more preferred embodiment, the high pressurized container (4) is a canister.

It is an object of the invention, a valve mechanism (25) is configured for immediately releasing only the gas form from the valve mechanism (25) upon actuation, at a pressure equal to the liquid propellant's vapor pressure. The valve mechanism (25) comprises a valve housing (22) and a valve stem (6). In a preferred embodiment, the level of the liquefied propellant in liquid form is below the tip of the valve housing (22) where an exit orifice (24) is positioned and/or the tip of a dip tube (26) affixed to the tip of the valve housing (22). In a preferred embodiment, no exit orifice (24) of the valve housing (22) and/or the tip of a dip tube (26) affixed to the tip of the valve housing (22) is dipped in the liquid form of a liquid propellant. In another preferred embodiment at least an orifice (24) is positioned on the side of the valve housing (22). The orifice (24) enables extra propellant only from the gas phase (headspace) to exit from the valve stem (6). Both embodiments are illustrative and not restrictive. Both preferred embodiments can be combined to form a single embodiment. While the two specific embodiments have been described, it is understood that the present invention can be applied to a wide variety of valve mechanisms (25) which immediately release only the gas form from the valve mechanism (25) upon actuation, at a pressure equal to the liquid propellant's vapor pressure.

Within the scope of this invention is the valve mechanism (25) actuation. Preferably, the actuation of the valve mechanism (25) comprises applying pressure at the bottom of the high-pressure container (18). Upon actuation, a precise amount of the liquid propellant in gas form is released.

The apparatus (1) comprises, a single mixing chamber (23) formed by compartment (20) of the capsule holder (2) with the substantially U-shaped compartment (9) of the container holder (3) when interlocked. Preferably, the single mixing chamber (23) is substantially spherical. In the single mixing chamber (23) the powder composition from the hard capsule (21) is mixed with the stream of the propellant gas that is released from the high pressurized container (4), upon valve mechanism (25) actuation. Preferably, the single mixing chamber (23) formed by compartment (20) of the capsule holder (2) with the substantially U-shaped compartment (9) of the container holder (3) when interlocked, is coated with a coating material. More preferably the coating material is based on polyurethane acrylic. The coating material is used to reduce friction losses of the powder and the propellant's gas form mixture.

Within the scope of this invention, the minimum distance of the vertex of the substantially U-shaped compartment (9) of the container holder (2) to any point of the surface of the capsule holder compartment (20) when interlocked, is determined by the hard capsule (21) size. If the minimum distance is equal or less than the length of the hard capsule body (17), the hard capsule body (17) may lodge either during hard capsule (21) breakage or upon valve mechanism (25) actuation. The single mixing chamber (23) dimensions influence the hard capsule body (17)/gas flow interactions and the powder transport efficiency. The larger the minimum distance, the greater is the gas flow fraction participating in the rotational mixing inside the single mixing chamber (23). However, the increase in rotational flow inside the chamber is coupled with a reduction in the flow velocity at the outlet which is detrimental to the Venturi suction effect. Preferably, the minimum distance of the vertex of the substantially U-shaped compartment (9) of the container holder (3) to the capsule holder compartment (20) when interlocked, is more than the length of the hard capsule body (17) and less than 15% of the length of the hard capsule body (17).

In another embodiment, the inlet gas orifice (7) is placed off-centre (offset). In this embodiment, the propellant in gas form is diverted to the hard capsule body (17) creating a relatively large vortex effect and hence a tumbling motion of the hard capsule body (17). In said embodiment with the offset inlet gas orifice (7), large rotational oscillations occur, which lead to emptying of the dose. In a preferred embodiment, the inlet gas orifice (7) is centrally located at the vertex of the substantially U-shaped compartment (9) of the container holder (3). The main mechanism of emptying the hard capsule (21) in the preferred embodiment is the Venturi effect, which is effective for powder transport.

The apparatus (1) comprises capsule inlet opening (10) configured to retain at least partially a hard capsule (21). It is an object of the invention to hold and break open a hard capsule (21) for releasing a powder formulation.

In one embodiment, the capsule inlet opening (10) configured to retain at least partially a hard capsule (21) is conical frustum shaped. In a preferred embodiment the conical frustum shaped opening (10) is circular at the entrance of the capsule inlet opening (10), configured to retain at least partially, the hard capsule cap (16) and rectangular shaped at the exit of the capsule inlet opening (10) communicating with the single mixing chamber (23). The circular entrance of the capsule inlet opening (10) facilitates the partial insertion of the hard capsule (16) into the single mixing chamber. Furthermore, the exit of the capsule inlet opening (10) is rectangular shaped. The rectangular shaped exit of the capsule inlet opening (10) facilitates the breaking-open of the hard capsule (21) without deforming the hard capsule body (17). Preferably, the rectangular shaped exit of the capsule inlet opening (10) consists of chamfered corners. The chamfered corners ensure an air-tight seal of the hard capsule cap (16) with the single mixing chamber (23), when the hard capsule (21) is broken-open.

In one embodiment of the invention, means for releasing the powder composition from the hard capsule is mounted internally into the U-shaped compartment (9) of the container holder (3). The hard capsule (21) is forced open into the single mixing chamber (23) formed by compartment (20) of the capsule holder (2) with the substantially U-shaped compartment (9) of the container holder (3) when interlocked. In a preferred embodiment the means for releasing the powder composition from the hard capsule (21) comprise shear-force opening means, needle-piercing means and cutting of the capsule means. In a more preferred embodiment, a breakage fin (8) is mounted internally into the U-shaped compartment (9) of the container holder (3).

The breakage fin (8) disengages the hard capsule body (17) from the hard capsule cap (16) placed in the capsule inlet opening (10) by shear-force action enabling the powder to be released from the hard capsule (21). In a further embodiment the breakage fin (8) is mounted internally into the U-shaped compartment (9) of the container holder (3) is configured to apply the shearing force 1 to 2 mm away from the bottom end of the hard capsule body (17). Application of the shearing force 1 to 2 mm away from the bottom end of the hard capsule body (17), minimizes hard capsule (21) deformation, which could lead to inefficient powder transport.

An embodiment of the invention is a powder delivery apparatus (1) for topical administration to a body orifice or a mucous membrane thereof of powder from a hard capsule (21) comprising:
a) a container holder (3) comprising two compartments configured to communicate through an inlet gas orifice (7); wherein a substantially U-shaped compartment (9) is configured to be mounted at the capsule holder (2) by interlocking snapping means (13) on the external surface of the substantially U-shaped compartment (9) and a breakage fin (8) mounted internally into the substantially U-shaped compartment (9) and a second compartment (19) configured to retain the high pressure container valve mechanism (25) tip at the one end of an inlet gas orifice (7);
b) a capsule holder (2) comprising an outlet nozzle (11), a compartment (20) configured to form a single mixing chamber (23) with the substantially U-shaped compartment (9) of the container holder (3) when interlocked, for powder composition and gas to be mixed; and a capsule inlet opening (10) configured to retain at least partially a hard capsule (21); c) an applicator (5) configured to be mounted on the outlet nozzle (11) for delivering powder from a hard capsule (21) to a body orifice or a mucous membrane
d) a high-pressure container (4) comprising a valve mechanism (25) comprising a valve housing (22) and a valve stem (6), configured to be safely secured to the container holder (3), wherein the valve mechanism (25) immediately releases only the gas form of a propellant contained in the high-pressure container (4) upon actuation, at a pressure equal to the propellant's vapor pressure.

### BRIEF DESCRIPTION OF THE FIGURES

FIG. 1 Side view of the assembled device.

FIG. 2 Side cross-sectional view of the assembled device without a hard capsule.

FIG. 3 Side view of the assembled device with a hard capsule.

FIG. 4 Exploded side view of the device.

FIG. 5 Exploded isometric view of the device.

FIG. 6 Side cross-sectional view of the device upon actuation (left) and detailed view of the mixing chamber (9) upon actuation (right).

FIG. 7 Side cross-sectional view of the device with capsule (left) and detailed view of the mixing chamber (9) with capsule (right).

FIG. 8 Side cross-section of the device in locked position with valve housing (22) with an exit orifice positioned at the tip of the valve housing (22) with a dip tube (26) (right) and with three exit orifice (24) positioned on the side of the valve housing (22) (right).

FIG. 9 A perspective view of a valve housing (22) with an exit orifice positioned at the tip (left) and with three exit orifices (24) positioned on the side of the valve housing (22) (right).

FIG. 10 Graph of the recorded temperature upon actuation versus distance from the valve mechanism point.

FIG. 11 Graph of the relationship between powder mass discharge from hard capsule (21) and single mixing chamber (23) diameter.

### DETAILED DESCRIPTION OF THE INVENTION

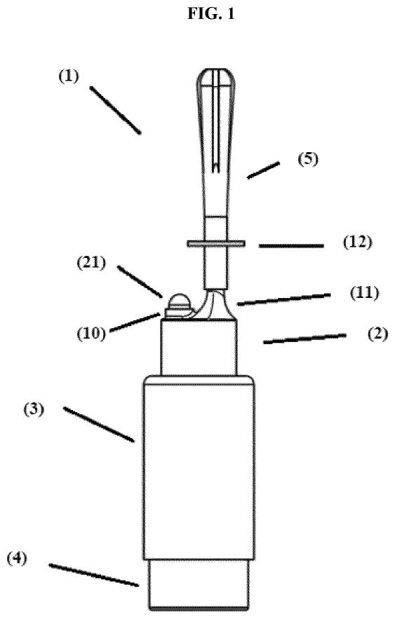

The invention is illustrated by, but not limited to, the following description of preferred embodiments of the invention. The present invention relates to a powder delivery apparatus (1) for topical administration of powder from a hard capsule (21) comprising a high pressure container (4) containing of a propellant(s) in liquid and in gas form, equipped with a valve mechanism (25) comprising a valve housing (22) and a valve stem (6), configured to be safely secured to the container holder (3) and for immediately releasing only the gas form from the valve mechanism (25) upon actuation, at a pressure equal to the liquid propellant's vapor pressure. As shown particularly in FIG. 1-FIG. 7 the apparatus (1) comprises a high-pressure container (4) equipped with a valve mechanism (25).

In a preferred embodiment the invention the capsule holder (2) and the container holder (3) are two interlocking plastic injection moulded components. The powder delivery apparatus (1) for topical administration is designed to receive and break open a powder-filled hard capsule (21).

The capsule holder (2) is interlocked to the container holder (3) via snapping means (13) to restrict detachment during operation while allowing the rotation of the capsule holder (2) around the container holder (3). A hard capsule (21) consisting of a powder composition is inserted into the capsule inlet opening (10) of the capsule holder (2). When the capsule holder (2) is rotated around the container holder (3), the hard capsule body (17) contacts a sharp protrusion, a hard capsule breakage fin (8) which is mounted into the substantially U-shaped compartment (9) of the container holder (3). The rotating action generates a shear force which is sufficient to break open the hard capsule (21) shell, separating the capsule body (17) from the capsule cap (16). The hard capsule cap (16) seals the capsule inlet opening (10), during use. A high velocity powder plume is obtained by combining the powder composition from the hard capsule body (17) and the propellant in gas form released form the high-pressure container (4) via the inlet gas orifice (7) upon actuation, in the single mixing chamber (23). The stream of the propellant in gas form in the single mixing chamber (23) vacates the hard capsule body (17) from the powder by vibration. The turbulent high velocity powder plume is directed to the outlet nozzle (11) of the capsule holder (2), being the only exit of the mixing chamber (23), when interlocked and in use. The applicator (5) is fitted on the outlet nozzle (11) and directs the powder plume topically. The applicator (5) is inserted into the body cavity and the stopper (12) prevents insertion to inappropriate depth.

In a first embodiment, FIG. 1 illustrates the side view of the assembled apparatus. The apparatus is shown as ready to use, where the capsule holder (2) is snapped on to the container holder (3). Furthermore, the hard capsule (21) is inserted into the capsule inlet opening (10) and the high-pressure container (4) adjusted and retained in the container holder (3). The applicator (5) with a stopper (12), is fitted on the outlet nozzle (11) of the capsule holder (2), ready to deliver topically the powder from a hard capsule (21), at the appropriate depth.

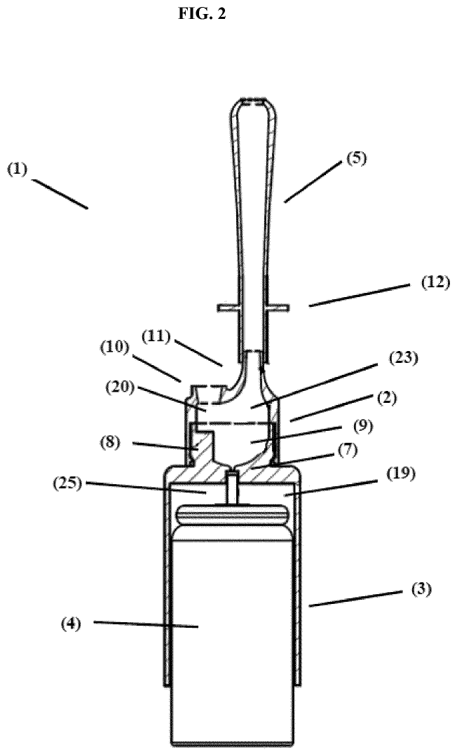

FIG. 2 shows a side cross-sectional view of the assembled apparatus without the hard capsule (21), which shows the position of the valve mechanism (25) of a high-pressure container (4) inside the second compartment (19) of the container holder (3). In the illustrated embodiment the inlet gas orifice (7) is centrally placed at the vertex of the substantially U-shaped compartment (9) of the container holder (3). The side cross-sectional view of the assembled device shows the side view of the breakage fin (8), which is mounted internally into the U-shaped compartment (9) of the container holder (3). The single mixing chamber (23) formed by compartment (20) of the capsule holder (2) with the substantially U-shaped compartment (9) of the container holder (3) when interlocked is shown. The embodiment illustrates the applicator (5) configured to be mounted on the outlet nozzle (11) based on the capsule holder (2). Furthermore, in one of the embodiments, the applicator (5) may be inserted into the vaginal cavity and the stopper (12) indicates the insertion depth. The conical frustum shaped inlet opening (10) is shown.

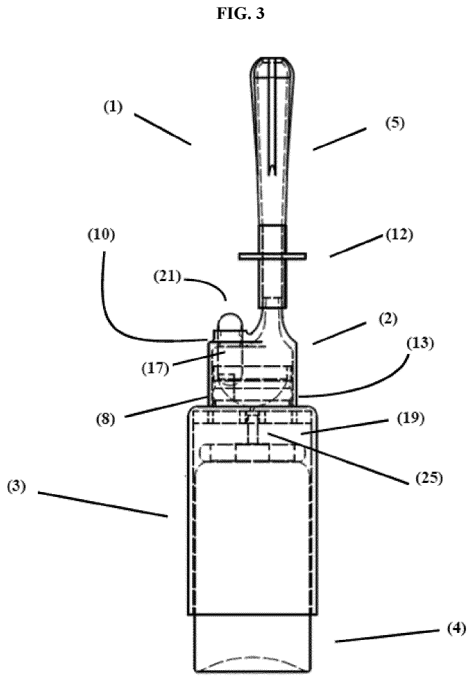

In FIG. 3 the side view is showing the insertion depth of the hard capsule (21) inside the inlet opening (10) such that the capsule body (17) contacts the capsule breakage fin (8) when the capsule holder (2) is rotated around the snapping means (13). Again, the container holder (3) is shown to retain the high-pressure container (4) by the valve mechanism (25) at the second compartment (19). The applicator (5) with the stopper (12) is shown.

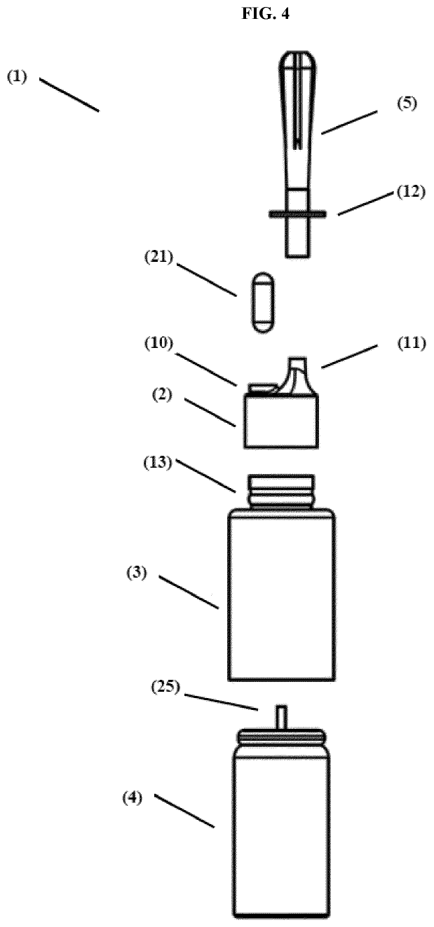

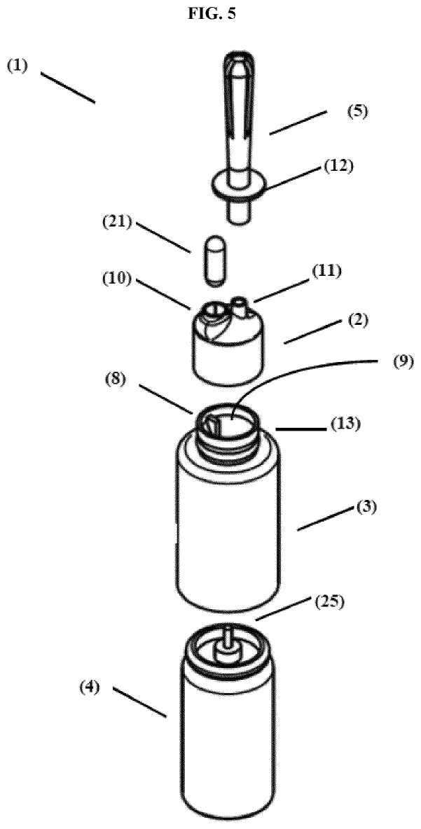

An exploded side view of the apparatus (1) is shown in FIG. 4. In the embodiment, the main components prior to assembly are a hard capsule (21), a capsule holder (2), a container holder (3) with snapping means (13), a high-pressure container (4) and the applicator (5) with a stopper (12). The outlet nozzle (11) and the capsule inlet opening (10) on the capsule holder (2) and the valve mechanism (25) on the high-pressurized container (4) are shown. An exploded isometric view of one of the embodiments is shown in FIG. 5. A hard capsule (21), the capsule holder (2) with an outlet nozzle (11), the container holder (3) with snapping means (13), a high-pressure container (4) and the applicator (5) with a stopper (12) are shown, before assembly. The breakage fin (8), which is mounted internally into the U-shaped compartment (9) of the container holder (3) and the inlet opening (10) configured to retain at least partially a hard capsule (21) at the capsule holder (2) are shown. Also, an external view of the valve mechanism (25) of the high-pressure container (4) is shown.

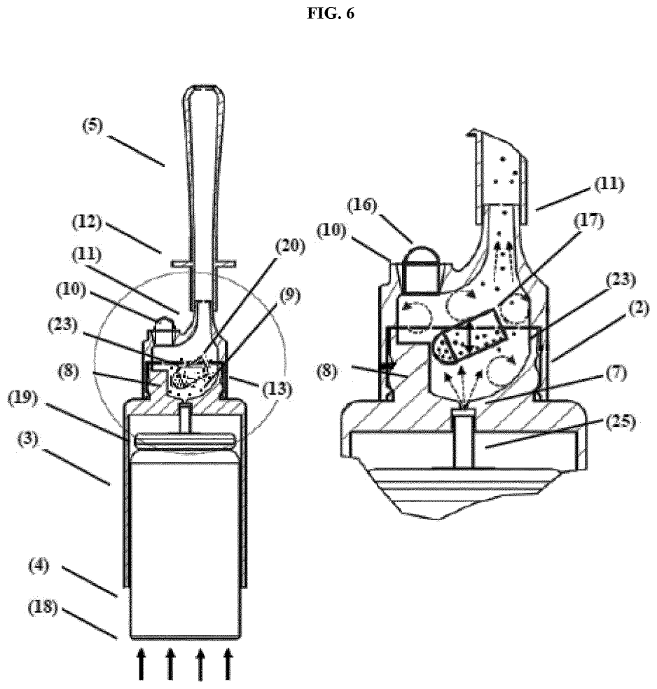

In further detail of the embodiment on the left of FIG. 6, the single mixing chamber (23) formed with the substantially U-shaped compartment (9) of the container holder (3) when interlocked is shown in FIG. 6, without a hard capsule (21) placed in the capsule inlet opening (10). The capsule holder (2) is tightly attached to the container holder (3) via snapping means (13). Furthermore, the actuation of the valve mechanism (25), is illustrated by applying pressure at the base (18) of the high-pressure container (4). The high-pressure container (4) is securely retained in the second compartment (19) of the container holder (3). Again, the applicator (5) placed on the outlet nozzle (11) of the capsule holder (2) equipped with a stopper is shown. The detailed view (right) illustrates an amount of the liquid propellant in gas form being released from the valve mechanism (25) and entering the single mixing chamber (23) formed by compartment (20) of the capsule holder (2) with the substantially U-shaped compartment (9) of the container holder (3) when interlocked, from the chamber inlet gas orifice (7), and the powder composition from the hard capsule (21) placed in the high-pressure container (4) being released after breakage of the hard capsule (21) by the breakage fin (8). The capsule's body (17) interacts with the propellant's gas form in the single mixing chamber (23) whereas the capsule cap (16) seals tight the inlet opening (10).

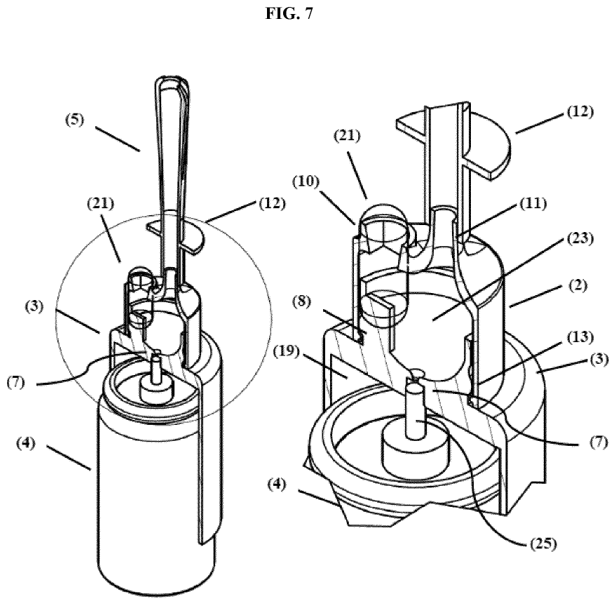

In FIG. 7, a cross-section of the device in locked position, is illustrative of the single mixing chamber (23) formed by compartment (20) of the capsule holder (2) with the substantially U-shaped compartment (9) of the container holder (3) when interlocked. Also, the conical frustum shaped capsule inlet opening (10) which is circular at the entrance of the inlet opening (10), configured to retain the hard capsule cap (16) and rectangular shaped at the exit of the inlet opening (10) communicating with the single mixing chamber (23), as shown. The inlet gas orifice (7) is shown with the tip of the valve mechanism (25) of a high-pressure container (4) retained in the container holder (3), being placed in the second compartment (19) of the container holder (3). Again, the breakage fin (8) and the applicator (5) placed on the outlet nozzle (11) equipped with a stopper are shown.

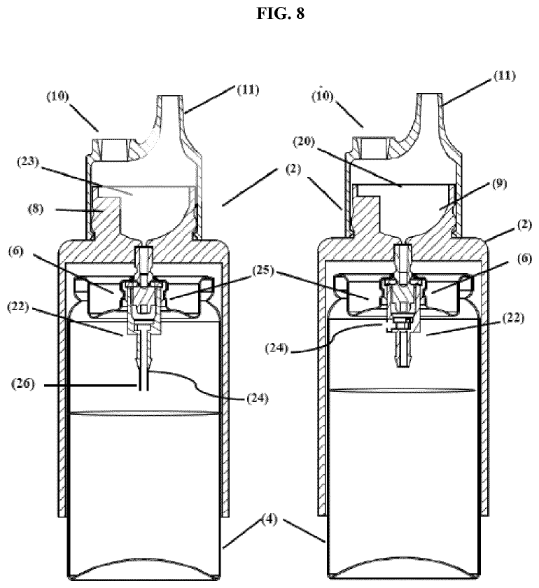

In FIG. 8 two embodiments of the invention are shown. The apparatus (1) is assembled, and the single mixing chamber (23) formed by compartment (20) of the capsule holder (2) with the substantially U-shaped compartment (9) of the container holder (3) when interlocked, is shown. Also, the capsule inlet opening (10) and the outlet nozzle (11) on the capsule holder (2) communicating with the single mixing chamber (23), are shown. The breakage fin (8) is also shown mounted in the substantially U-shaped compartment (9) of the container holder (3). Furthermore, the two embodiments illustrate the two possible configurations of the valve mechanism (25), comprising a valve stem (6) and a housing (22) with exit orifices (24). On the left embodiment the valve housing (22) comprises an exit orifice (24) positioned at the tip of the valve housing (22), whereas on the left embodiment an exit orifice (24) is positioned on the side of the valve housing (22). The level of the liquefied propellant in liquid form is below the exit orifices (24). A dip tube (26) extending valve housing (22) of the left embodiment may be attached to the tip of the valve housing (22) but the exit orifice of the dip tube (26) is again above the level of the propellant's liquid form. Embodiments are illustrative and not restrictive.

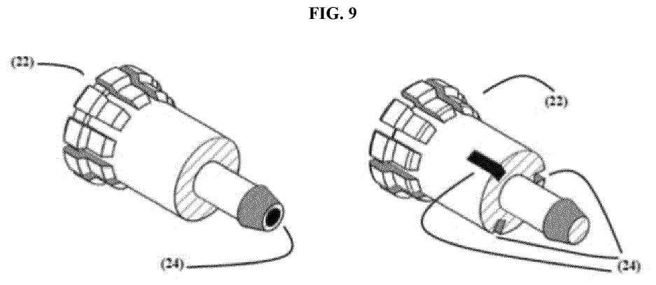

In FIG. 9, the configuration of a valve housing (22) with an exit orifice positioned at the tip of the valve housing (22) and a valve housing (22) with three exit orifice (24) positioned on the side of the valve housing (22) are illustrated.

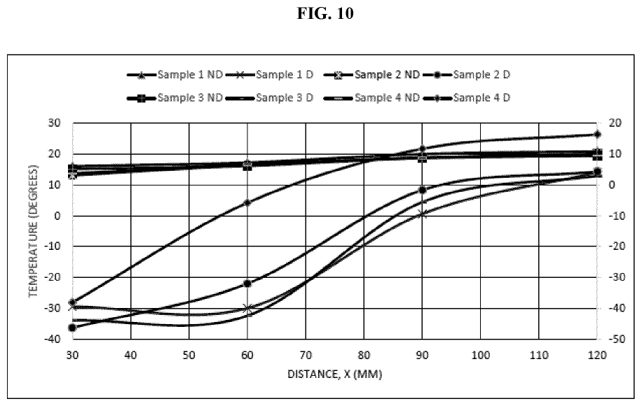

In FIG. 10 the graphical representation of the recorded temperature upon actuation versus distance from the valve mechanism (25) exit point at the distance of 30, 60, 90 and 120 mm, as per Example 1. The recorded temperature observed at −40 °C, is evident that the powder composition should not be dispersed in the liquid phase of the propellant. The invention's apparatus (1) is clearly diversified from the classical heterogenous aerosol systems by eliminating the ‘cold freon effect’.

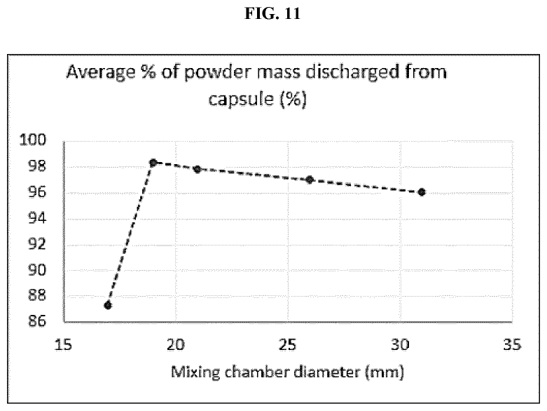

In FIG. 11 the graphical representation of the relationship between powder mass discharge from hard capsule (21) and single mixing chamber (23) diameter as per Example 3 is illustrated.

### EXAMPLES

The invention is described with reference to the following examples. This invention is not limited to these examples and experiments. Many variations will suggest themselves and are within the full intended scope of the appended claims. Nonlimiting selection of the valve mechanisms which were used in the following examples are shown in Table 1. In the C valve housing (22), the exit orifices (24) are located on the side of the housing body. The exit on the tip of the valve housing orifice is blocked and non-functional. Distance from slot to the bottom of the canister is 50 mm.

In the A configuration, the side exit orifice (24) is almost similar with the C, however the exit orifice (24) on the tip of the valve housing is fully functional. Distance from slot to the bottom of the canister is 43 mm.

Selected valve stem (6)s are illustrative and not restrictive.

TABLE 1
Valve mechanism comprising selection of
valve housing (22) and valve stem (6)
Valve Housing (22)

| | | Exit orifice | Exit orifice(s) | |
|---|---|---|---|---|
| | | positioned | positioned | Total |
| | | to tip of the | on the side of | inlet |
| Configu- | Diameter | valve housing | the valve housing | area |
| ration | (inch) | (22) (mm) | (22) (mm) | (mm2) |
| A | 1 | 1.6 | 0.6 | 2.29 |
| B | 1 | 2.0 | — | 3.14 |
| C | 1 | — | 3 slots of 3 × 1 mm | 9.00 |

Valve stem (6)

| | | Number | Inlet orifice | Outer |
|---|---|---|---|---|
| | | of inlet | (stem) | diameter |
| Configuration | Characteristic | orifices | diameter (mm) | (mm) |
| I | Sliding | 2 | 0.5 | 3.5 |
| | actuation | | | |
| II | Upside- down | 2 | 0.7 | 4 |
| | version | | | |

### Example 1

In-vitro measurements of HFA's 134a spraying plume's temperature confirmed that the temperature drop (ΔT), when using a valve drawing from the propellant's gas phase, is approximately 10-15 times less, compared to when using a valve housing (22) with a dip-tube (26) that draws from the propellant's liquid phase. The performance was the same throughout all distance points and up to 120 mm from the valve mechanism (25) point.

TABLE 2
Average temperature drop results of various valve samples tested with and
without a dip-tube, in four different distance points from the actuator.

| | | | Propellant HFA 134a | | | |
|---|---|---|---|---|---|---|
| | | | Average temperature drop difference (T1 − T2), ΔT (°C) | | | |
| Valve | Dip | | Distance A | Distance B | Distance C | Distance D |
| samples | tube | Draw point | x = 30 mm | x = 60 mm | x = 90 mm | x = 120 mm |
| Sample 1 ND | No | Vapor phase | 3.20 | 2.44 | 0.96 | 0.45 |
| Sample 1 D | Yes | Liquid Phase | 45.56 | 44.51 | 19.64 | 6.97 |
| Sample 2_ND | No | Vapor phase | 5.30 | 3.04 | 1.40 | 0.60 |
| Sample 2 ND | Yes | Liquid Phase | 54.32 | 47.81 | 12.04 | 6.26 |
| Sample 3 ND | No | Vapor phase | 3.62 | 2.94 | 1.20 | 0.61 |
| Sample 3 D | Yes | Liquid Phase | 54.10 | 49.89 | 16.50 | 7.68 |
| Sample 4 ND | No | Vapor phase | 3.30 | 1.63 | 0.78 | 0.34 |
| Sample 4 D | Yes | Liquid Phase | 57.14 | 26.00 | 7.73 | 3.98 |

### Example 2

The risk of drawing liquefied propellant from below the surface fill level was tested. The test was carried out to evaluate the volumetric expansion rate of the liquefied propellant when exposed to temperatures higher than the room temperature of 20 °C.

Enforced clear glass vials of known dimensions were used and filled with a predetermined quantity of liquefied HFA 134a at 5 bars pressure at 20 °C. The fill level height of the propellant inside the vial was measured each time (mm), upon exposure of the samples for 24 h to various temperatures.

TABLE 3
Propellant HFA 134a volume expansion test values (in terms of liquefied
propellant fill level) upon exposure to various temperatures.

| | | | | | | | | Total | |
|---|---|---|---|---|---|---|---|---|---|
| | | Level | Level | Level | | Level | | expansion | |
| | | of HFA | of HFA | of HFA | | of HFA | | difference | % Total |
| | Pressure | 134a at | 134a at | 134a at | % diff. | 134a at | % diff. | from 4 °C | difference |
| Bottle | at 20 °C | 20 °C | 30 °C | 40 °C | of 40 °C | 4 °C | of 4 °C | to 40 °C | from 4 °C |
| Number | (bar) | (mm) | (mm) | (mm) | vs 20 °C | (mm) | vs 20 °C | (mm) | to 40 °C |
| 1 | 5 | 36.23 | 36.15 | 38.87 | 7.21 | 35.5 | −2.01 | 3.37 | 9.30 |
| 2 | 5 | 36.06 | 37.09 | 38.69 | 7.29 | 35.41 | −1.80 | 3.28 | 9.09 |
| 3 | 5 | 38.87 | 39.75 | 40.97 | 5.40 | 36.61 | −5.81 | 4.36 | 11.21 |
| 4 | 5 | 37.18 | 37.94 | 40.27 | 8.31 | 37.04 | −0.37 | 3.23 | 8.68 |
| 5 | 5 | 37.01 | 37.88 | 39.37 | 6.37 | 35.74 | −3.43 | 3.63 | 9.80 |
| 6 | 5 | 37.77 | 39.87 | 40.9 | 8.28 | 39.63 | −2.22 | 3.97 | 10.51 |

| | | | |
|---|---|---|---|
| Average | 7.16 | Average | 9.77 |

It is evident that the propellant's volume expansion even after exposure at 40 °C is minimal. Therefore, there is no risk associated with drawing liquefied propellant from the surface level, as the average maximum difference from 20 °C to 40 °C is around 2.5 mm or 7.1%.

### Example 3

Fill both hard capsule (21) sizes (capsule sizes (#1 and #0)) with a pharmaceutical formulation of a powder presenting acceptable powder flow characteristics and having a PSD value of d90=20-25 am.

The proposed composition in Table 4.

TABLE 4
A proposed composition

| Excipients | Function |
|---|---|
| Lactose Anhydrous | Filler |
| Polycarbophil USP | Film former |
| Carbomer Homopolymer Type B | Film former |
| Amorphous Colloidal Silicon Dioxide | Glidant |
| Talc, purified | Glidant |
| Magnesium Stearate veg. | Lubricant |

The moisture content of the powder composition inside the hard capsule (21), should be well below 4%.

The hard capsule's (21) mass load:
Hard capsule (21) size #0=Loaded with 65 mg of powder composition
Hard capsule (21) size #1=Loaded with 32 mg of powder composition

Fill canisters with HFA 134a at a gauge pressure of 5 bars at 20 °C and crimp them employing the following valve configurations:
C slotted valve housing (22), I version stem
B valve housing (22), I version stem

Apparatus (1) characteristics:
Single mixing chamber (23) diameters: 17 mm, 21 mm, 26 mm, 31 mm
Inlet gas orifice (7) position: Centre, Offset
Inlet gas orifice (7) diameters: 1 mm, 1.5 mm

TABLE 5
Mass loss test to evaluate valve performance. Test was performed with hard capsule (21) Size #1.

| | | | | Average of | Average of | |
|---|---|---|---|---|---|---|
| | | Standard | Average of | remaining | remaining | |
| | | deviation | powder mass | powder | powder | Average |
| | Average of | of powder | discharged | mass on | mass on | mass of |
| | powder mass | mass | from hard | hard capsule | container | propellant |
| | discharged | discharged | capsule | (21) holder | holder | discharged |
| Valve type | (%) | (%) | (21) (%) | (2) (%) | (3) (%) | (g) |
| I valve stem (6) | 82.45 | 5.82 | 96.87 | 7.07 | 5.84 | 1.50 |
| B valve housing (22) | | | | | | |
| Dip tube YES | | | | | | |
| I valve stem (6) | 80.66 | 7.13 | 96.32 | 5.19 | 8.54 | 1.82 |
| B valve housing (22) | | | | | | |
| Dip tube NO | | | | | | |
| I valve stem (6) | 84.52 | 3.68 | 96.41 | 4.49 | 6.64 | 2.40 |
| C valve housing (22) | | | | | | |
| Dip tube NO | | | | | | |

The results show that the best performance is achieved with the C valve housing (22) and the I valve stem (6) combination. Said combination has the largest opening area (3 slots of 3×1 mm slits in housing) which results in the highest propellant flow rate.

TABLE 6
Mass loss test to evaluate optimum single mixing chamber (23)
diameter. Test was performed with Hard capsule (21) Size #1

| | | | | | Average | Average of | Average of | |
|---|---|---|---|---|---|---|---|---|
| Single | | | | Standard | of powder | remaining | remaining | |
| mixing | | Inlet gas | | deviation | mass | powder mass | powder | Average |
| chamber | Inlet gas | orifice | Average of | of powder | discharged | on hard | mass on | mass of |
| (23) | orifice | (7) | powder mass | mass | from hard | capsule (21) | container | propellant |
| diameter | (7) | diameter | discharged | discharged | capsule | holder (2) | holder | discharged |
| (mm) | position | (mm) | (%) | (%) | (21) (%) | (%) | (3) (%) | (g) |
| 17 | centre | 1 | 85.06 | 14.74 | 87.31 | 0.20 | 0.94 | 2.39 |
| 19 | centre | 1 | 93.09 | 3.68 | 98.34 | 1.45 | 3.49 | 2.17 |
| 21 | centre | 1 | 89.72 | 2.51 | 97.83 | 2.29 | 4.69 | 3.12 |
| 26 | centre | 1 | 88.92 | 3.82 | 96.99 | 2.69 | 4.86 | 2.99 |
| 31 | centre | 1 | 86.81 | 2.91 | 96.03 | 1.86 | 6.19 | 3.06 |

TABLE 7
Mass loss test to evaluate optimum inlet gas orifice (7) diameter
and position. Test was performed with Hard capsule (21) Size #1

| | | | | | | Average % | Average % | | |
|---|---|---|---|---|---|---|---|---|---|
| Single | | | | | | of powder | of remaining | Average % | |
| mixing | | Inlet gas | | Average | Standard | mass | powder | of remaining | Average |
| chamber | Inlet gas | orifice | | Percentage | deviation | discharged | on hard | powder on | Mass of |
| (23) | orifice | (7) | | of powder | of powder | from hard | capsule (21) | container | propellant |
| diameter | (7) | diameter | | discharged | discharged | capsule | holder (2) | holder (3) | discharged |
| (mm) | position | (mm) | | (%) | (%) | (21) (%) | (%) | (%) | (g) |
| 21 | centre | 1 | | 89.72 | 2.51 | 97.83 | 2.29 | 4.69 | 3.12 |
| 21 | centre | 1.5 | | 89.7 | 1.79 | 96.74 | 2.71 | 3.66 | 3.17 |
| 21 | offset | 1 | | 84.78 | 4.92 | 96.55 | 5.61 | 5.56 | 3.064 |
| 21 | offset | 1.5 | | 86.14 | 6.1 | 97.76 | 6.02 | 5.60 | 3.13 |
| 19 | centre | 1 | mm | 93.09 | 3.68 | 98.34 | 1.45 | 3.49 | 2.17 |
| 19 | centre | 1.5 | mm | 94.66 | 3.23 | 97.67 | 0.66 | 2.05 | 3.11 |

The centered inlet gas orifice (7) is more effective for powder transport.

Moreover, a marginal increase in performance is obtained with a larger inlet gas orifice (7) diameter, however this entails a considerable increase in propellant discharge and hence decrease in high pressure container lifetime.

TABLE 8
Mass loss test to compare performance between hard capsule (21) Size #0 and hard capsule (21) Size #1

| | | | | | | Average | Average of | | |
|---|---|---|---|---|---|---|---|---|---|
| | | | | | | of | remaining | | |
| | | | | | Standard | powder | powder | Average | |
| | | | | Average | deviation | mass | mass on | of | |
| | Single | | Inlet | of | of | dis- | hard | remaining | |
| | mixing | Inlet | gas | powder | powder | charged | capsule | powder | Average |
| | chamber | gas | orifice | mass | mass | from | (21) | mass on | mass of |
| Hard | (23) | orifice | (7) | dis- | dis- | hard | holder | container | propellant |
| capsule | diameter | (7) | diameter | charged | charged | capsule | (2) | holder | discharged |
| size | (mm) | position | (mm) | (%) | (%) | (21) (%) | (%) | (3) (%) | (g) |
| 0 | 21 | center | 1 mm | 94.35 | 2.45 | 98.52 | 1.56 | 2.57 | 3.16 |
| 1 | 19 | center | 1 mm | 93.09 | 3.68 | 98.34 | 1.45 | 3.49 | 2.17 |

Sufficient propellant flow rate is available to levitate hard capsule (21) size 0 and transport a larger powder dose. The performance is slightly improved compared to the 33 mg tests because the amount of powder which is deposited on the single mixing chamber (23) surface is not directly proportional to the initial powder mass.

### Example 4

The high-pressure container comprises HFA134a and HFO1234ze. Both liquid propellants provide comparable excellent results. The configuration used in the example is shown:
Device: container holder (2) M4_D44a, capsule holder (2): M6_D55b;
Device coating: Coating based on polyurethane acrylic;
Container valve: Valve stem (6): II;
Valve housing (22): C;
Hard capsule (21) size #0;
Target dose: 66 mg

TABLE 9
Mass loss test to compare performance between HFA134a and HFO1234ze

| | | | | | Average | |
|---|---|---|---|---|---|---|
| | | Standard | | Average | remaining | Average |
| | Average | deviation | Average | powder mass | powder | remaining |
| | powder mass | of powder | propellant | discharged | mass on | powder mass |
| | discharged | mass | mass | from hard | container | on capsule |
| | from device | discharged | discharged | capsule | holder (3) | holder (2) |
| Propellant | (%) | (%) | (g) | (21) (%) | (%) | (%) |
| HFA134a | 94.73 | 1.68 | 2.62 | 98.35 | 1.42 | 1.47 |
| HFO1234ze | 94.62 | 1.13 | 2.02 | 97.97 | 0.94 | 0.81 |

1. A powder delivery apparatus (1) for topical administration to a body orifice or a mucous membrane thereof, of a powder composition from a hard capsule (21) comprising:
a) the container holder (3) comprising two compartments configured to communicate through an inlet gas orifice (7); wherein a substantially U-shaped compartment (9) is configured to be mounted at the capsule holder (2) by interlocking snapping means (13) on the external surface of the substantially U-shaped compartment (9) and means for releasing the powder composition from the hard capsule (21) mounted internally into the substantially U-shaped compartment (9) and a second compartment (19) configured to retain the high pressure container valve mechanism (25) tip at the one end of an inlet gas orifice (7);
b) a capsule holder (2) comprising an outlet nozzle (11), a compartment (20) configured to form a single mixing chamber (23) with the substantially U-shaped compartment (9) of the container holder (3) when interlocked, for powder composition and gas to be mixed; and a capsule inlet opening (10) configured to retain at least partially a hard capsule (21);
c) optionally, an applicator (5) configured to be mounted on the outlet nozzle (11) and
d) a high-pressure container (4) comprising a valve mechanism (25) comprising a valve housing (22) and a valve stem (6), configured to be safely secured to the container holder (3), wherein the high-pressure container contains a liquid propellant in liquid and in gas form for immediately releasing only the gas form from the valve mechanism (25) upon actuation, at a pressure equal to the liquid propellant's vapor pressure.

2. A powder delivery apparatus (1) according to claim 1, wherein the high-pressure container (4) consists essentially of a liquid propellant in liquid and in gas form.

3. A powder delivery apparatus (1) according to claim 1, wherein the liquid propellant is selected from 1,1,1,2-tetrafluoroethane, 1,3,3,3-Tetrafluoropropene and mixtures thereof.

4. A powder delivery apparatus (1) according to claim 1, wherein all the exit orifice (24) of the valve housing (22) is dipped in the gas form of a liquid propellant.

5. A powder delivery apparatus (1) according to claim 1, wherein at least one an exit orifice (24) is positioned on the side of the valve housing (22).

6. A powder delivery apparatus (1) according to claim 1, wherein the level of the liquefied propellant in liquid form is below the exit orifice (24) on tip of the valve housing (22) or the exit orifice (24) on the tip of a dip tube affixed to the tip of the valve housing (22).

7. A powder delivery apparatus (1) according to claim 1, wherein the actuation of the valve mechanism (6) involves applying force to the base of the container (18)

8. A powder delivery apparatus (1) according to claim 1, wherein a high-pressure container is a metallic canister or enforced glass vial.

9. A powder delivery apparatus (1) according to claim 1, wherein the capsule inlet opening (10) is conical frustum shaped.

10. A powder delivery apparatus (1) according to claim 1, wherein the applicator (5) has stopper (12) indicating the insertion depth into a body orifice.

11. A powder delivery apparatus (1) according to claim 10, wherein the applicator (5) is of single or multi-use.

12. A kit comprising i) an apparatus according to claim 1, ii) at least one hard capsule comprising a powder composition in a blister card or a container, iii) an applicator (5) and iii) an instruction leaflet for use.

13. A kit according to claim 12, wherein the powder composition is a pharmaceutical powder composition.

14. A method for delivering powder composition from a hard capsule (21) to a body orifice or a mucosal membrane thereof, comprising the steps of i) providing an apparatus (1) according to claim 1, ii) assembling the apparatus (1) of step i), iii) providing a hard capsule (21) prefilled with powder composition iv) twisting the container holder (3) against the capsule holder (2), iv) placing the outlet nozzle (11) or the applicator (5) in the body orifice or a mucous membrane v) actuate the valve mechanism (25).
